(19) United States
(12) Patent Application Publication
(10) Pub. No.: US 2012/0021445 A1
(43) Pub. Date: Jan. 26, 2012

(54) Pluripotent Stem Cells for Drug Induced Myopathy and Malignant Hyperthermia

(76) Inventors: Kirk J. Hogan, Madison, WI (US); Hector H. Valdivia, SR., Middleton, WI (US)

(21) Appl. No.: 13/171,745

(22) Filed: Jun. 29, 2011

Related U.S. Application Data
(60) Provisional application No. 61/359,650, filed on Jun. 29, 2010.

(51) Int. Cl. C12Q 1/50; C12Q 1/02

(52) U.S. Cl. 435/17; 435/29

(57) ABSTRACT

The present invention relates to methods of identifying, collecting and isolating pluripotent stem cells, and compositions of purified stem cells for the diagnosis of susceptibility to drug induced myopathy (DIM) and malignant hyperthermia (MH). Specifically, the present invention provides methods of producing skeletal muscle myocytes from patient-specific stem cells, and methods for identifying susceptibility to DIM and MH in patient-specific skeletal muscle myocytes generated from patient-specific stem cells. The present invention also relates to methods, compositions and kits for screening drugs for DIM and MH risk.

### FIELD OF THE INVENTION

The present invention relates to methods of identifying, collecting and isolating pluripotent stem cells, and compositions of purified stem cells, for the diagnosis of susceptibility to drug induced myopathy (DIM) and malignant hyperthermia (MH). Specifically, the present invention provides methods of producing skeletal muscle myocytes from patient-specific stem cells, and methods for identifying susceptibility to DIM and MH in patient-specific skeletal muscle myocytes generated from patient-specific stem cells. The present invention also relates to methods, compositions and kits for screening drugs for DIM and MH risk.

### BACKGROUND OF THE INVENTION

Adverse drug reactions (ADRs) account for approximately 5% of all hospital admissions and 5% of all fatalities. (Klopstock T. Curr Opin Neurol 2008;21:590-595). Skeletal muscle is susceptible to DIM because it is exposed to circulating drugs. DIM is defined as the presence of myopathic symptoms and signs, including muscle weakness, fatigue, muscle pain, creatine kinase (CK) elevation or myoglobinuria, that occur in patients without or without pre-exisitng muscle disease when exposed to certain drugs. (Dalakas M C. J Neurol Neurosurg Psychiatry 2009;80:832-838). After discontinuation of the drug, the signs of muscle involvement often improve in support of the causal effect of the inciting myotoxic drug. On occasion the toxicity is irreversible e.g., fialuridine, a nucleoside analogue that causes irreversible myocytotoxicity by incorporating into the mitochondrial DNA chain. A muscle biopsy is useful to diganose myotoxicity, but may be uninformative, as seen in a subset of patients with myoglobinuria or mild muscle weakness caused by statins or nucleoside analogues. Moreover, a muscle biopsy is invasive, disfiguring, costly, risky and limited in the number of studies to be performed. Histological changes observed in toxic myopathies vary from non-specific alterations to necrotizing, inflammatory or vacuolar myopathy. Because toxic myopathies are potentially reversible, rapid diagnosis and is needed to begin therapy, prevent irreversible muscle changes and thwart secondary effects in other tissues, for example, the kidney.

### SUMMARY OF THE INVENTION

The present invention relates to methods of identifying, collecting and isolating pluripotent stem cells, and compositions of purified stem cells for the diagnosis of susceptibility to drug induced myopathy (DIM) and malignant hyperthermia (MH). Specifically, the present invention provides methods of producing skeletal muscle myocytes from patient-specific stem cells, and methods for identifying susceptibility to DIM and MH in patient-specific skeletal muscle myocytes generated from patient-specific stem cells. The present invention also relates to methods, compositions and kits for screening drugs for DIM and MH risk.

In some embodiments, the present invention provides a method for identifying susceptibility to drug induced myopathy and/or malignant hyperthermia in a subject, comprising: providing a sample from a subject containing one or more cells; producing one or more stem cells from the one or more cells; generating one or more skeletal muscle myocytes from the one or more stem cells; testing the one or more skeletal muscle myocytes for susceptibility to drug induced myopathy and/or malignant hyperthermia; and identifying the subject as susceptible or not susceptible to drug induced myopathy and/or malignant hyperthermia based on the results of the testing of the one or more myocytes. In certain embodiments, the subject is a mammal. In other embodiments, the mammal is a human. In further embodiments, the sample is skin sample, a cheek swab sample, a mouthwash sample, a muscle sample, a blood sample, or a cord blood sample. In still further embodiments the one or more cells is a fibroblast, a keratinocyte, an adipose cell, a nucleated blood cell, a muscle cell, or a mucosal cell. In additional embodiments, the stem cell is embryonic stem cell. In preferred embodiments, the stem cell is a construct of a nucleus from one or more cells from a subject and a host cell. In particularly preferred embodiments the stem cell is an induced pluripotent stem cell (iPSC). In some embodiments, testing comprises testing in the presence and the absence of a drug.

In some embodiments, the present invention provides a method that comprises gene transfer. In other embodiments, the method comprises administration of one or more growth factors, epigenomic agonists, epigenomic antagonists, or small molecules. In further embodiments, the testing comprises imaging the one or more skeletal muscle myoctes. In still further embodiments, the testing comprises cell electrophysiology. In certain embodiments, the cell electrophysiology comprises measuring one or more responses to electrical stimulation, chemical stimulation (for example, potassium or acetylcholine), fractional shortening, or twitch tension. In some embodiments, the testing comprises testing mitochondrial integrity. In other embodiments, the testing comprises testing mitochondrial function. In further embodiments, the testing comprises testing mitochondrial intracellular constitutents. In other embodiments, the testing comprises measuring [³H]ryanodine binding to solubilized induced pluripotent stem cell skeletal muscle myocyte homogenates. In further embodiments, the testing comprises direct determination of RyR1 activity. In still further embodiments, the testing comprises measuring [Ca²⁺]ᵢ transients in induced pluripotent stem cell skeletal muscle myocytes. In preferred embodiments, the testing comprises measuring the viability of induced pluripotent stem cell skeletal muscle myocytes in the presence of RyR1 agonists and antagonists. In particularly preferred embodiments, the testing comprises measuring a sarcoplasmic Ca²⁺ leak-load relationship. In some embodiments, the testing comprises the extracellular measurement of an intracellular compound comprising protons, lacatate, creatine kinase, myoglobin, troponin, or a metabolite. In other embodiments, the testing comprises the intracellular measurement of an intracellular compound comprising calcium, magnesium, ATP, lactate, fatty acids and fatty acid esters. In further embodiments, the testing comprises microscopy, for example light microscopy, confocal microscopy, laser microscopy, electron microscopy, and sample handling with specific reagents and dyes before microscopy.

In some embodiments the present invention provides a method for screening a drug for drug induced myopathy or malignant hyperthermia risk, comprising: providing s drug; providing one or more skeletal muscle myocytes produced from one or more induced puripotent stem cells from one or more subjects susceptible to drug induced myopathy and/or malignant hyperthermia; applying the drug to the one or more myocytes; testing the myocytes for susceptibility to drug induced myopathy and/or malignant hyperthermia; and identifying the drug as having drug induced myopathy and/or malignant hyperthermia risk based on the results of the testing of the one or more myocytes.

In some embodiments the present invention provides a method for the manufacture of at least one bioinformatic descriptors of use in identifying susceptibility to drug induced myopathy and/or malignant hyperthermia in a subject, comprising: providing a sample from a subject containing one or more cells; producing one or more stem cells from the one or more cells; generating one or more skeletal muscle myocytes from the one or more stem cells; testing the one or more skeletal muscle myocytes for susceptibility to drug induced myopathy and/or malignant hyperthermia; and identifying the subject as susceptible or not susceptible to drug induced myopathy and/or malignant hyperthermia based on the results of the testing of the one or more myocytes. In certain embodiments, the subject is a mammal. In other embodiments, the mammal is a human. In further embodiments, the sample is skin sample, a cheek swab sample, a mouthwash sample, a muscle sample, a blood sample, or a cord blood sample. In still further embodiments the one or more cells is a fibroblast, a keratinocyte, an adipose cell, a nucleated blood cell, a muscle cell, or a mucosal cell. In additional embodiments, the stem cell is embryonic stem cell. In preferred embodiments, the stem cell is a construct of a nucleus from one or more cells from a subject and a host cell. In particularly preferred embodiments the stem cell is an induced pluripotent stem cell (iPSC). In some embodiments, testing comprises testing in the presence and the absence of a drug.

In a further embodiment, provides testing for variations introduced in the process of producing the stem cells including testing for genetic polymorphisms, copy number variations, and epigenomic variations. In other embodiments, testing comprises genetic rescue of the DIM/MH phenotype as a control comprising, for example, removal of a mutant allele, overexpression of a wild-type allele, or replacement of a mutation by gene targeting. In still further embodiments, test results in skeletal muscle iPSCs from subjects having, or suspected of having, DIM and/or MH are compared to test results from DIM and/or MH skeletal muscle iPSCs with and without genetic modification from other DIM and/or MH subjects, to test results from skeletal muscle iPSCs with and without genetic modification from normal control subjects, and to test results from skeletal muscle iPSCs from one or more family members with and without DIM and/or MH.

### DETAILED DESCRIPTION

The present invention relates to methods of identifying, collecting and isolating pluripotent stem cells, and compositions of purified stem cells, for the diagnosis of susceptibility to drug induced myopathy (DIM) and malignant hyperthermia (MH). Specifically, the present invention provides methods of producing skeletal muscle myocytes from patient-specific stem cells, and methods for identifying susceptibility to DIM and MH in patient-specific skeletal muscle myocytes generated from patient-specific stem cells. The present invention also relates to methods, compositions and kits for screening drugs for DIM and MH risk.

### Drug Induced Myopathy (DIM)

Clinical signs and symptoms of DIM are variable and include myalgia, hyperCKemia (hyper-creatinine kinase-emia), muscle weakness or myoglobinuria. According to the type of injury to the muscle fibre or specific organelle, the toxic myopathies may be classified as:

1. Necrotising myopathy caused, for example, by statins, fibrates and epsilon aminocaproic acid (EACA), is defined by scattered necrotic fibres invaded by macrophages in the absence of MHC-I up-regulation or a large number of lymphocytic infiltrates invading non-necrotic fibres.
2. Inflammatory myopathy with features similar to polymyositis, including CD8+ T cells invading non-necrotic, MHC-I-expressing fibres, caused by statins, d-penicillamine, and alpha interferon, or intramuscular injections of genes.
3. Thick filament loss myopathy seen in critical care, often caused by steroids in the setting of acute denervation or in combination with neuromuscular blocking agents.
4. Type II muscle fibre atrophy most commonly caused by steroids combined with inactivity.
5. Mitochondrial myopathy characterised by the presence of “ragged red” or “ragged blue” fibres, COX-negative fibres and increased lipid accumulation, and seen most often with nucleoside analogues.
6. Lysosomal storage myopathy caused by amphiphilic drugs (e.g., chlorochine), which contain a hydrophobic region that interacts with acidic phospholipids of membranes, generating the storage of myeloid structures within the lysosomes.
7. Anti-microtubule myopathy most often caused by colchicine, which inhibits polymerisation of microtubules and causes disruption of cytoskeletal network with swollen lysosomes and autophagic vacuoles.
8. Myofibrillar myopathy often caused by emetin which disrupts of tZ discs followed by breakdown of myofilaments and accumulation of myofibrillar proteins.

Drugs implicated in toxic myopathy include: anticholesterol statins (cerivastatin>simvastatin>atarvastatin>lovostatin>pravastatin>fluvastatin), concomitant drugs increasing the risk of statin-associated myopathic symptoms (fibrates (especially gemfibrozil)) but also clofibrate or niacin); cyclosporine; azole antifungals; macrolide antibiotics; HIV-protease inhibitors; nefrazodone; verapramil; amiodarone; antirheumatic/inflammatory/immunosuppressive drugs d-penicillamine, colhicine, chlorochine, α-interferon, ciclosporin, tacrolimus, steroids; antinucleoside analogues zidovudine, fialuridine; contaminated products for example, 1-tryptophan contaminants;

aluminium-containing vaccines; toxic oils; dietary agents germanium, emetin; recreational cocaine, heroin, amphetamines, PCP, alcohol; antipsychotics, epsilon aminocaproic acid, procainamide, succinylcholine, volatile anesthestics (for example, halothane, isoflurane, disflurane, sevoflurane), and amiodarone (when combined with statins). As new drugs are introduced, this listing will increase.

### Anticholesterol: Statin-Induced Myopathies

Statins are a group of fungus-derived drugs that inhibit the 3-hydroxy-3-methyl-glutaryl-coenzyme A (HMG-CoA) reductase, the enzyme that catalyses the conversion of HMG-CoA to mevalonic acid, the precursor of cholesterol. Statins affect the mitochondria and the sarcoplasmic reticulum especially in the type II fibres which contain 30% less fat than the type I fibres, rendering them more vulnerable to damage caused by a reduction in cholesterol available for membrane biosynthesis. Dysfunction of mitochondria has been shown in muscle biopsies of patients with statin-induced myopathic symptoms. Some patients may have elevation of CK that does not exceed five to six times the ULN, rarely reaching up to 10 times the ULN. These patients have normal strength and no complaints of fatigue or muscle pain. Muscle pain is reported in up to 9-25% of statin-treated patients and usually improves after discontinuation of the drug. In other patients a myopathy is observed. In some the myopathy is mild, subacute and temporally related to the initiation of statin therapy; in others the myopathy is chronic. Muscle biopsies may show necrotic fibres without inflammation. Statins are the most effective medications for managing elevated concentrations of low-density lipoprotein cholesterol (LDLc), and accordingly are among the most prescribed drugs in the world. Statins reduce morbidity in both coronary heart disease patients, and in previously healthy subjects. Over 40 million Americans have high cholesterol; 20 million are currently prescribed a statin class of cholesterol lowering drugs. DIM is the primary clinical management challenge of statins, particularly when treatment targets require LDL cholesterol levels below 100 mg/dl. DIM is more prevalent at the higher doses required for treating advanced cardiovascular disease and diabetes, and varies widely between individual statins and from patient to patient. Statin usage is ultimately limited by toxicity. DIM requires alteration of therapy, burdens healthcare with management costs, and reduces compliance. Only 50% of patients remain on statins 6 months after initiation of therapy.

Rhabdomyolysis is defined as an acute elevation of CK (creatinine kinase) (>15 000 ULN) that may be accompanied by myalgia, weakness and myoglobinuria. Its reported incidence varies, from 0 for fluvarastatin, 0.04 for pravastatin and atrovarstatin, 0.19 for lovostatin to as high as 3.16 for cervistatin. FDA data estimate that the incidence of rhabdomyolysis is similar for all statins when used as monotherapy. The incidence of rhabdomyolysis is increased when statins are combined with other drugs including amiodarone, gemfibrozil or cyclosporine. Statins are metabolised to varying degrees by CYP 3A4, except pravastatin which is cleared by the kidneys. Concomitant medications may inhibit CYP3A4, thereby increasing the concentration of statins. The risk of rhabdomyolysis is exacerbated by other factors liver failure, hypothyroidism and diabetes. Predisposition to statin myopathy is linked to alleles of the SLCO1B gene that encodes an organic anion transporter. Rhabdomyolysis is a medical emergency that requires immediate discontinuation of drugs together with hydration and electrolyte control. Because the drugs must be combined with others with DIM risk in certain settings (e.g, statins with fibrate or niacin in patients with a very high level of LDL-C, statins with cyclosporine in transplant recipients, statins with antiretrovirals in HIV-positive patients), caregivers must be aware of increased risks and monitor the patients closely. 60% of patients with simvastatin myopathy are attributed to variant SLCO1B1, hence testing for homozygosity for the risk allele c may reduce the incidence of statin myyopathy.

### Antinucleoside Analogues

Patients treated with the nucleoside analogue reverse transcriptase inhibitors (NRTIs) may develop a myopathy after long-term therapy. Zidovudine (AZT) causes myopathy; stavudine (d4T) and fialuridine (FIAU) cause neuropathy or myopathy and lactic acidosis. The tissue distribution of phosphorylases responsible for phosphorylation of NRTIs relates to their selective tissue toxicity. The clinical features of zidovudine myopathy are proximal muscle weakness, occurring 6-12 months after treatment onset, myalgia (predominantly in the thighs and calves), fatigue, myopathic changes on EMG and elevated serum CK levels, that may increase with exercise. Weight loss and elevation of the serum lactate level may denote the onset of zidovudine myopathy. When zidovudine is discontinued, symptoms resolve, muscle histology improves, and the depleted mtDNA rebounds. AZT-treated patients demonstrate high lactate production and marked phosphocreatine depletion, as determined by in vivo MRS spectroscopy arising from impaired oxidative phosphorylation. Animals treated with AZT and cells in culture treated with NRTIs develop similar changes.

### Recreational Drugs

Cocaine, heroin, amphetamine, PCP and ETOH may cause rhabdomyolysis. Patients with alcoholism may develop an acute or a chronic myopathy. The acute myopathy presents as rhabdomyolysis or myoglobinuria, and may be preceded by muscle edema and pain. It can recur if the patient resumes drinking Acute myopathy in patients with alcoholism may also be related to hypokalaemia when the serum K⁺ concentration is below 2.5 mEq. This myopathy is painless, not accompanied by muscle swelling and is rapidly reversible. Although the exogenous causes of myoglobinuria may be multifactorial, illegal drugs and alcohol account for the majority of the patients.

### Other

Procainamide, amiodarone, epsilon-aminocaproid acid (EACA) and antipsychotics can cause myopathic symptoms. Even in the absence of neuroleptic malignant syndrome, antipsychotics may cause hyperCKaemia. Up to 10% of the patients receiving clozapine, risperidone, melperone, olanzapine, loxapine or haloperidol may develop CK elevation. Sometimes the CK may be above 1000. Muscle biopsy may be uninformative. Because these drugs are potent serotonin 5-HT2α inhibitors, and 5HT receptors are present in the sarcolemma of skeletal musce, the toxic effect may be related to blockade of these receptors. Hundreds of other drugs can cause muscle damage. Myotoxic reactions may not be due to single drugs but to drug-drug interactions. The most prominent and clinically relevant examples are any combinations of statins, fibrates, cyclosporine, and protease inhibitors. Many myotoxic events could be avoided by careful choice of drugs or dose adjustment. On the contrary, withdrawal of statins because of asymptomatic creatine kinase elevation or mild and tolerable myalgias is not necessary and may be dangerous given the undisputed benefit of statins for cardiovascular endpoints.

### Malignant Hyperthermia (MH)

Hereditary rhabdomyolysis associated with administration of volatile anesthetic agents and depolarizing muscle relaxants is a pharmacodynamic toxic response. Currently, the Malignant Hyperthermia Association of the United States (MHAUS) is notified of about 200 probable trigger events per year, with deaths unfortunately part of each annual report. It is estimated that 3-400 additional unreported cases of MH occur each year. “Malignant hyperthermia” (MH) i.e., muscle destruction and consequent hyperkalemia, acidosis, dysrhythmia, renal failure and disseminated intravascular coagulation after parenteral succinylcholine or inhalation of potent anesthetic vapors, is fatal in a significant proportion of patients despite early detection and appropriate management including provision of dantrolene. The tragedy is compounded by an otherwise full life expectancy in the absence of exposure to trigger anesthetics. The lack of efficacy for dantrolene in up to 5% of malignant hyperthermia patients despite a deepening understanding of its mechanism of action, suggests that pharmacogenetic markers for this antidote itself remain to be discovered.

All volatile anesthetics, including newer agents sevoflurane and desflurane, are potential triggers of the syndrome, although they may not be as florid in their presentation as their predecessors. An increase in temperature is often a late or inconsistent manifestation of the disorder, and recrudescence of the syndrome may occur in up to 20% of clinical reactions. These observations point to the need for improved detection of susceptibility, for earlier diagnosis based on features other than temperature (e.g., unexplained hypercarbia and dysrhythymia), and for a lengthened monitoring interval and specific therapy well after a trigger arises.

For more than 30 years after its elucidation as a clinical entity, MH was ascribed to a heterogenous inventory of underlying mechanisms including catechol excess, nonspecific membrane defects and lipid theories. Fairhurst, in drawing attention to the similarity between MH and toxicity of the sap of the ryana tree in Trinidadian rats, provided the first clue implicating a receptor-mediated phenomenon. The observation was accorded little attention until all porcine MH triggers, and a subset of human MH events, were linked to mutations in the skeletal muscle calcium release channel, which also came to be known as the ryanodine receptor (RYR1). Currently, more than 200 RYR1 polymorphisms have been found in patients manifesting the MH trait by clinical event or skeletal muscle contracture testing (in vitro contracture test (IVCT), or caffeine halothane contracture test (CHCT)), each with varying degrees of causal certainty. Polymorphisms in the coding regions of RYR1 are found in 50%-70% of patients with malignant hyperthermia, leaving 30% or more of predisposing mutations to be discovered elsewhere in RYR1 or its surrounding genome. At least 5 other susceptibility loci have been identified, with 6 mutations detected to date in the L-type calcium channel gene CACNA1S.

Accordingly, human malignant hyperthermia (MH) is a clinical syndrome, not a single disease, triggered by exposure to volatile anesthetics and depolarizing skeletal muscle relaxants that is often, but not always, inherited as an autosomal dominant trait with incomplete penetrance and variable expressivity. MH may trigger in the absence of specific drug exposure in response to, for example, heat stroke, heat exhaustion, and heavy exercise in high ambient heat environments. MH arises from mutations that exhibit both locus heterogeneity (i.e., MH from different genes), and allelic heterogeneity (i.e., MH from different mutations in a single gene). A substantial number of predisposing myopathies and conditions are recessively inherited, and de novo mutation has been described. Additionally, compound heterozygosity for RYR1 polymorphisms is increasingly reported in patients surviving clear-cut triggers, and discordance between genotype and phenotype has been observed. Although the incidence of clinical malignant hyperthermia varies between 1/15,000 to 1/50,000 anesthetics, the proportion of individuals in a given population with predisposing mutations may be 1/2000 or greater. Based on expression of malignant hyperthermia in mice transfected with a human RYR1 malignant hyperthermia-associated mutation, the causality of at least one RYR1 polymorphism in humans is no longer in doubt. However, the disproportion between the incidence of predisposing RYR1 alleles in the general population, and the apparent rarity of the full-fledged syndrome, points to additional acquired and inborn susceptibilities that have yet to be disclosed.

While genetic testing for MH differential diagnosis and counseling is minimally invasive, requiring only a few drops of blood or a cheek swab for DNA isolation, is easily replicated and reproduced, and is less costly than contracture testing depending on the number of mutations sought, inherent deficiencies also constrain widespread MH genotyping. Even with full length RYR1 sequencing, a number of mechanisms for MH genetic pathology may be missed, for example, copy number variations, epigenomic modifications, and polymorphisms, and alternate splice sites in unsequenced introns and promoter regions. Most importantly, a full length genomic characterization of RYR1 leaves the contribution of other known and suspected loci unscreened. Polymorphisms at MH loci may themselves act as Mendelian determinants of MH susceptibility, or they may serve in concert with known loci to amplify MH-susceptibility. Thus, in the setting of both locus and allelic genetic heterogeneity, interpretation of MH genetic test data is not straightforward. While the presence of a shared MH mutation in family members of a proband who has had a clear cut clinical MH trigger permits identification of these individuals as MH susceptible, the absence of one or more genotypes in question from a partial panel (i.e., a negative genetic test result) is not interpretable.

Independent of a clinical event, the most widely accepted method for ascertaining MH susceptibility is the ex vivo muscle biopsy configured for physiological testing of changes in contractility in the presence of volatile anesthetics (i.e., halothane) and the skeletal muscle Ca²⁺ release channel/ryanodine receptor (RYR1) agonist, caffeine. The tests, developed with different protocols in North America and Europe, are known as the Caffeine-Halothane Contracture Test (CHCT) and the In-Vitro Contracture Test (IVCT), respectively. Muscle bundle stimulated contracture strength thresholds that segregate normal, susceptible and equivocal (in Europe) responses, have been arrived at by consensus, based upon data from patients with clinically documented MH in the absence of coincident myopathy (which may itself introduce bias), and compared with the responses of normal individuals with an intentional bias favoring false positive designations in order to not miss potentially susceptible individuals if a higher cutoff is chosen.

The utility of the contracture test varies with its intended application i.e., for differential diagnosis of a confusing event in the operating room, for MH research, and as a guide to drug selection in clinical care and genetic counseling. For differential diagnosis, if positive results are found, thereby indicating that an untoward perioperative event was most probably MH, the contracture test holds substantial value and may be life-saving to a patient and family members. Negative results on a test performed for differential diagnosis are a greater challenge to interpretation, but may shift the focus away from a diagnosis of MH.

However, as an instrument for genetic counseling, and as an aid to the caregiver tasked with selecting a clinical anesthetic regimen in a relative of an MH patient, contracture testing has well-recognized shortcomings. Contracture testing is invasive, scarring, expensive (˜$5000), and is now performed at only five centers in North America. There is not, and cannot be, experimental validation of contracture test results on children below 10 years of age, a population at higher risk for MH than adults. The inter-laboratory replicability of the European IVCT (i.e., samples from the same patient tested in different laboratories using the same protocol) is not high (i.e., 56% between labs in the only published study). Comparable results using the North American CHCT protocol and thresholds have yet to appear. Nor has contracture test reproducibility (i.e., samples from the same patient tested in the same laboratory on different days) been reported for either the European or North American test. While a positive test result (and most probably an equivocal result) using whatever thresholds have been selected, provides evidence against the further use of trigger agents in a given patient or family member, the meaning of negative contracture tests in patients with enough indication to have the test in the first instance (e.g., the individual has a family member with MH) is not known. To measure the true incidence of false negative contracture test results requires sufficient numbers of IVCT/CHCT negative patients to undergo subsequent anesthetics with trigger agents. Since an MH susceptible individual may have as many as thirty uneventful anesthetics before experiencing a trigger, the number of anesthetics needed on contracture test-negative individuals in order to determine the false negative incidence with precision is unsettled. Moreover, the incidence of false negative (and false equivocal) results is likely to vary between distinct MH-predisposing mutations in distinct genetic backgrounds. In turn, different RYR1 mutations confer varying degrees of cellular dysfunction as measured by agonist-induced Ca²⁺ release in samples from patients with distinct genetic backgrounds, thereby undermining confidence that one or two threshold contracture test values are sufficient to segregate all susceptibility to the MH syndrome into two or three categories (i.e., negative, positive, and/or equivocal) for the purposes of genetic counseling. Therefore, no such investigation of the incidence of false negative contracture test responses has been, or is likely to be conducted, and no peer-reviewed scientific data is available to the concerned patient, family member, or caregiver, regarding the safety of administering drugs that trigger MH to patients who are contracture test negative (or equivocal).

To improve the genotype-phenotype correlation, and resolve the significant number of patients diagnosed as “MH equivocal”, a number of modifications of the contracture test have been proposed, with the addition of 4-chlorochresol appearing to hold the greatest promise. Although this alteration segregates many equivocal muscle bundles into either MH susceptible or MH normal groups, it remains premature to state whether the segregation is in fact correct (fully predictive of the clinical trigger phenotype), or to provide the precise false equivocal rate. Less invasive tests sufficiently sensitive to detect a single mutant RYR1 copy must be developed in humans and model organisms, thereby sharpening phenotypic indicators for use in humans at risk. Improved phenotypic and genotypic detection is particularly desired in view of growing recognition that MH-like events may occur in susceptible patients after anesthesia, or even in the absence of exposure to trigger drugs. These circumstances, coupled with limited lab-to-lab reproducibility of the contracture test, points to the great need for an improved malignant hyperthermia “endo-phenotype” interposed between DNA sequence variation and the clinical event based on in vivo contracture testing, whole muscle imaging, muscle cells grown in culture, or calcium kinetics in Epstein-Barr virus (EBV)-transformed lymphoblasts cultured from B-lymphocytes. For this to occur, however, a number of questions must first be answered. For example, while EBV-transformed cells have many attractive properties as test vehicles, it is not known whether the RYR1 channel expressed in lymphoblastoid cells is identically regulated in comparison to that of skeletal muscle. Nor is it known if the response of RYR1 in circulating white cells is a true surrogate for the MH responsiveness of skeletal myocytes.

To the contrary, stem cell and somatic nuclear transfer technology offers attractive solutions to these problems. For example, patient specific nuclei carrying mutations of interest may be isolated from such sources as white blood cells or skin fibroblasts, and transferred into pluripotential cells. In turn, the modified cells may then be induced to form muscle cells upon which exposure to trigger agents and physiological testing (for example, by measuring Ca²⁺ transients and acidification in response to 4-cmc) can proceed. As well, patient-specific induced pluripotent stem cells (iPSCs) may be generated from readily available tissues, and transformed into differentiated cell lines for specific characterization of their phenotypes. Thus, human genetic heterogeneity is amenable to quantification with rigorous control of the polymorphism of interest, the genetic background of the sample, and the cellular apparatus of the relevant differentiated cell. Correlation of data from these methods with detailed clinical information, genotyping, and with quantitative rather than categorical data derived from contracture testing underlies their validation and refinement. Since the mutation itself need not be known in order to discriminate modified cells from MH and non-MH sources, phenotypes resolved in this fashion are fitting substrates for pedigree, phylogenetic and genome-wise association studies (GWAS) aimed at discerning novel genetic loci capable of imparting MH-susceptibility.

### Pluripotent Stem Cells

Genetically modified or immortalized animal and human cell lines may be used to screen compounds for tissue specific toxicity. However, these cells may not provide an accurate representation of how various drug candidates initiate responses in humans and other organisms. Other challenges include biochemical differences between animal and human cells, and changes in cellular responses as a result of the immortalization of human cells to produce lines in culture. Moreover, it is desirable to screen drugs on a final tissue of interest, which is not often represented by currently used cell lines. Nevertheless, such immortalized cell lines are used for drug screening because they are cheap, easy to grow, reliable and reproducible.

Recent advances in stem cell research underlie alternative cell strategies. For example, adipose-derived stem cells (ASCs) obtainable under local anesthesia may be induced to differentiate into skeletal muscle cells. (Mizuno H. The potential for treatment of skeletal muscle disorders with adipose-derived stem cells. Curr Stem Cell Res Ther 2010; 5(2); 133-136) As well, embryonic stem cell (ESC) and induced pluripotent stem cell (iPSC) approaches provide a source of human cells that can be grown to quantities necessary for drug screening and toxicology studies. ESC and iPSC cells can then be differentiated to generate tissue-specific and patient-specific cell lines including skeletal muscle myocytes. (Mizuno et al. Generation of skeletal muscle stem/progenitor cells from murine and human induced pluripotent stem cells. (FASEB J Epub Feb. 24, 2010), Kazuki Y. et al. Complete correction of iPS cells from Duchene Muscular Dystrophy. Mol Ther 2010; 18(2): 386-393.) In turn, iPSCs may now be derived from patients with specific diseases, for example DIM and MH.

Human ESCs are derived from the inner cell mass of early stage fertilized embryos. ESCs continually self-renew to maintain the undifferentiated stem cell population, and retain the capacity to generate cell types in the 3 germ layers i.e., endoderm, mesoderm and ectoderm. Transcription factors and chemical induction signals needed for specializing human ESCs to form germ layers are now available. Accordingly, cells from a single source may be used to produce many of the cells within the human body that could be affected by a drug, providing an improved predictive model of toxicity than rodent cell lines or immortalized human cell lines. However, controversy surrounds use of human ESCs because embryos must be destroyed during the procurement. In turn, technical challenges confront expanding and differentiating PSCs in large numbers. Human ESCs are difficult to grow in comparison with typical cell lines requiring daily changes of growth medium and they are susceptible to spontaneous differentiation. In addition, human ESCs are predisposed to chromosomal abnormalities detected by serial karyotype screening. Reproducibility is hampered by variations in growth and differentiation properties arising among human ESC lines. Although techniques for generating purified populations of specialized tissues (for example, heart or brain) have been reported, standard, validated techniques are poorly established. Most protocols are variable, inefficient, expensive, and require long periods of time to produce the desired cell types. These limitations imply that the benefits of investing in human ESC technologies for drug screening may not justify the expense.

In some eombodiments, iPSCs are generated by expressing a limited set of pluripotency genes in somatic cells (e.g., fibroblasts, melanocytes, keratinocytes, mesenchymal cells, cord blood cells, adult blood cells, adipose cells, or other adult cell types) to trigger genetic reprogramming. The reprogrammed cells lose previous somatic properties and adopt characteristics to human ESCs in morphology, growth, gene-expression and differentiation. Conversion of mouse somatic cells into iPSCs has been accomplished using four genes: Oct4_(also known as Pou5f1), Sox2, Klf4 , and c-myc_(also known as Myc). Others have reprogrammed human and rodent somatic cells into iPSCs using modified techniques and pluripotency genes (NANOG, LIN28). Additional methods include use of non-integrating viruses, episomes, or plasmid transfection, proteins, small-molecule induction (e.g., DNMT and histone deacetylase inhibitors) and complementation that generate stable cell lines. Methods for producing iPSCs may be found, for example, in Thomson et al. Somatic Cell Reprogramming (U.S. Patent Publication No. 20110028537, filed Mar. 18, 2010), Thomson et al. Pluripotent Stem Cells Obtained by Non-Viral Preprogramming (U.S. Patent Publication No. 20100184227, filed Oct. 23, 2009, Amada et al. Methods for the Production if OIPS Cells Using Non-Viral Approach (U.S. Patent Publication No. 20100003757, filed Jun. 4, 2009), Dimos et al. Methods and Compositions for Selection of Stem Cells (U.S. Patent Publication No. 20110029814, filed Jun. 4, 2010), Yamanaka et al. Methods of Efficiently Establishing Induced Pluripotent Stem Cells (U.S. Patent Publication No. 20110039338, filed Jul. 30, 2009), Yamanaka et al. Method of Preparing Induced Pluripotent Stem Cells Deprived of Reprogramming Gene (U.S. Patent Publication No. 20110003365, filed May 28, 2010), Yamanaka, Nuclear Reprogramming Factor and Induced Pluripotent Stem Cells (U.S. Patent Publication No. 20100216236, filed Feb. 18, 2010) and Yamanaka et al. Efficient Method for Nuclear Reprogramming (U.S. Patent Publication No. 20100075421, filed Nov. 28, 2008), each of which is incorporated herein in its entirety . Unlike human ESCs, human iPSCs over do not require human embryo destruction, and many iPSC lines can be established from a single donor and tests results compared between one another. As well, iPSCs may be derived from patients and probands after a disease has been diagnosed and its phenotype (e.g., drug triggers, severity, and age of onset) is known.

In certain embodiments of the present invention, differentiating the iPSCs are differentiated to cells of interest i.e., skeletal muscle myocytes, and then concentration-response toxicity assays are performed. In some embodiments, subjects with DIM and MH susceptibility are identified by abnormal responses to DIM and MH agonists and antagonists in cells with subject-specific mutations, genetic backgrounds, and tissue relevant morphology and physiology. In other embodiments, drugs are screened for the capacity to cause DIM and MH in cells from subjects with known susceptibility. In this fashion, cost-efficient, reliable, and simple screening reagents are produced that enable toxic compounds to be eliminated at an early stage of the drug discovery process, allowing efforts to be directed to more promising candidates. Provided with the appropriate nutrient-rich environment, iPSCs may be instructed to form the various cell types found in the human body, including myocytes, which are then used to assess multiple cellular characteristics. Lineage-restricted iPSCs are then used in the early stages of the drug discovery process to identify novel compounds, and to evaluate their safety and efficacy in specialized cell types. These preclinical results are then used to advance compounds through the next stages of drug development.

### Pharmacogenomic Drug Screening

Although iPSCs can be used to model both sporadic, epigenetic and genetic diseases, they are particularly advantageous for investigating diseases that have a pharmaocgenetic or pharmacoepigenetic background and/or occur early in development such as DIM and MH. A particular advantage of iPSC models of DIM and MH is that DIM and MH are expressed as cellular disorders, unlike conditions such as cardiac long-QT syndrome that are expressed at the whole tissue level, often in the setting of heterogenous cell populations.

Testing iPSC Myocytes for DIM and MH Susceptibility

### EXPERIMENTAL

The following experimental example are provided in order to demonstrate and further illustrate various aspects of certain embodiments of the present invention, and are not to be construed as limiting the scope thereof. In the experimental disclosure which follows, the following abbreviations apply: μg (micrograms); ng (nanograms); ml (milliliters); C (degrees Centigrade).

### Example 1

Skeletal muscle fibers are generated by activated satellite cells, the muscle-specific stem cells that differentiate into myoblasts and form myotubes to replace the myofibers damaged by exercise, daily activities and drugs. (Mizuno Y. The FASEB Journal 2010, published online Feb. 24, 2010). Embryonic stem cells (ESC) are attractive source of cells for DIM and MH diagnosis and drug screening due to their capacity for proliferation in an undifferentiated state over a prolonged period, and their ability to differentiate into various lineages of cells as observed in vivo. Rodent and human induced pluripotent stem cells (iPSCs) may be generated by transfecting pluripotency-associated genes into somatic cells.

Similar to ESCs, the re-programmed somatic cells self-renew and are pluripotent. iPSC methods are able to produce stem cells as a predicate to patient-oriented disease investigations and screening for drug toxicity. Muscle stem cells may be induced from ESCs cells in vitro, for example, by plating embryoid bodies (EBs) onto Matrigel-coated plates. Using Ab SM/C-2.6_myogenic lineages may be induced from iPSCs, thereby providing quantitative assays of mature and immature skeletal muscular lineage cells in vitro and in vivo.

### Materials and Methods

### Cell Lines

A 4-factor iPSC line reprogrammed by the introduction of Oct3/4, Sox2, Klf4, and c-Myc (clone 38D2), and a 3-factor iPSC line that lacks c-Myc (clone 256H-18), is produced from embryonic fibroblasts.

In Vitro Differentiation of ES Cells and iPSCs into a Muscle Cell Lineage

To delete feeder cells, undifferentiated ESCs and iPSCs are treated with 0.5% trypsin/ethylenediaminetetraacetic acid (Life Technologies, Inc., Grand Island, N.Y., USA) and transferred onto tissue culture dishes (Falcon; BD Biosciences, San Diego, Calif., USA) coated with 0.1% gelatin (Sigma-Aldrich, St. Louis, Mo., USA) in maintenance medium supplemented with 5000 U/ml leukemia inhibitory factor (LIF), at a concentration of 5_{—}103 cells/cm2. For embryoid body (EB) formation, ESCs and iPSCs are cultured in 3 d at a density of 800 cells/20_{—}1 differentiation medium, consisting of Dulbecco's modified Eagle medium (DMEM) supplemented with 10% fetal calf serum (FCS), 5% horse serum (Sigma), 0.1 mM 2-mercaptoethanol, 0.1 mM nonessential amino acid, and 50_g/ml penicillin/streptomycin. EBs are ttransferred to a suspension culture in the differentiation medium for an additional 3 d. Therefater, each EB is plated onto 48-well tissue culture plates (Falcon) coated with Matrigel Basement Membrane Matrix (BD Bioscience, Bedford, Mass., USA).

### RT-PCR Analysis

RNA isolation and RT-PCR are performed according to established protocols. Oligonucleotide primers are specific for Pax3, Pax7, MyJS, MyoD, Myogenin, and GAPDH.

Efficient Skeletal Muscle Differentiation from iPSCs In Vitro

Differentiation of skeletal muscle fibers from iPSCs in vitro is initiated after the initial 6 d in a hanging drop and suspension culture. EBs attach to the plates and, 7 d after plating onto the Matrigel-coated plates (d 13 of differentiation), spindle-shaped fibers appear. After 14 d of plating (d 20 of differentiation), tspindle fibers grow, migrate out of the EB, and fuse, forming multinucleated fibers with skeletal muscle morphology. In iPSC-derived cultures, GFP expression is not observed from d 13 of differentiation, indicating that the undifferentiated marker NANOG is inactivated during differentiation. Fibers begin spontaneous contraction on d 27 of differentiation, as seen in normal skeletal myofibers. RT-PCR is used to examine the expression of genes associated with skeletal myogenesis. Pax3 and Pax7 expressed selectively by satellite cells are essential for the specifying myogenic progenitors from the central dermomyotome. The stem/progenitor cells display myogenic properties by expressing muscle regulatory factors (MRFs), such as Myf5, MyoD, and myogenin. Expression of myogenic marker genes begins from d 13 of differentiation, peaking on d 20 of differentiation in both ESC and iPSC derived cultures. Immunostaining for Pax3, Pax7, Myf5, MyoD, and myogenin confirms skeletal muscular lineage cells on d 20 of differentiation.

Using a modified culture system to plate EBs onto Matrigel-coated dishes, the emergence and proliferation of mature skeletal myofibers is observed. Sequential RT-PCR detects PAX3 and PAX7 expression, followed by expression of myogenic markers including Myf5, MyoD, and myogenin indicating that myogenic markers areinvolved during iPSC differentiation as in normal myogenesis. The anti-satellite cell Ab SM/C-2.6 is used to enrich for skeletal muscle stem/progenitor cells in the iPSC differentiation systems. Selection using SM/C-2.6 Ab reduces teratoma formation.

### MH Testing in Myocytes

A characteristic of MH is an abnormal intracellular Ca²⁺ homeostasis triggered by hyperactivity of RyR1. A variety of functional assays that specifically track the activity of RyR1 are used. For example, the activity of RyR1 at the molecular level is determined. iPSC-derived skeletal myocytes (iPSC-SkM) from normal and MH-susceptible individuals are used to obtain whole cell homogenates and sarcoplasmic reticulum (SR)-enriched microsomes. Each functional assay requires only a small amount of RyR1 protein (1-10 μg). Intrinsic alterations elicited by MH mutations are determined using a combination of biochemical and electrophysiological techniques including: 1.) Ca²⁺-dependence of [³H]ryanodine binding to solubilized iPSC homogenates in the absence and the presence of RyR1 modulators (caffeine, 4-chlorochresol, halothane, and scorpion venom); and 2.) reconstitution of sarcoplasmic reticulum vesicles in planar lipid bilayers with recording of single RyR1 channel activity in the absence and the presence of Ca2+ and caffeine. RyR1 is also tested for hyperactivity in cellular experiments. In multiple cells, 3.) the amplitude and duration of the intracellular Ca²⁺ ([Ca²⁺]ᵢ) transient in response to chemical depolarization (incremental concentrations of [K⁺]ₒ), or MH triggers (volatile anesthetics, succinylcholine), or agonists of RyR1 (caffeine, 4-chorochresol, scorpion venom) is determined. As well, 4.) the viability of iPSC-SkMs in the continued presence of RyR1 agonists, and 5.) the SR Ca²⁺ leak-load relationship, which is high in MH, are quantified. Thus, concentration-response curves are generated that differ between control and MH-susceptible individuals, and the risk of developing MH episodes based on RyR1 hyperactivity is identified.

1. [³H]Ryanodine Binding to Solubilized iPSC-SkM Homogenates

Ligands that enhance Ca²⁺ release from the SR enhance [³H]ryanodine binding, whereas ligands that inhibit Ca²⁺ release inhibit [³H]ryanodine binding, indicating that [³H]ryanodine binds to a conformationally-sensitive domain of the RyR1. The binding of [³H]ryanodine is thus an index of the activity (open probability, Pₒ) of the RyR1. The intrinsic activity of RyR1, as well as its response to MH triggers (caffeine, volatile anesthetics) in detergent-solubilized iPSC-SkMs homogenates from normal and MH-susceptible individuals, is determined. A standard incubation medium consisting of 0.2 M KCl, 10 mM Na-PIPES, pH 7.2, 0.1% CHAPS, 7 nM [³H]ryanodine and 0.5 mg/ml protein is used for all samples. Free [Ca²⁺] is fixed in the range of 0.1 nM to 100 μM by adding 1 mM EGTA and variable concentrations of CaCl₂ to the incubation medium. MH trigger drugs are added from concentrated stocks (>100-fold) and the effect of the vehicle, if any, is subtracted. Incubation lasts 90 min at 36° C. Under these conditions, the association reaction of [³H]ryanodine to solubilized receptors is ˜95% complete. Samples (0.1 ml) are run in duplicate, filtered onto glass fiber filters, and washed twice with 5-ml cold water. Non-specific binding is determined with 20 μM “cold” ryanodine and is subtracted from the binding curves. Specific binding is plotted against free [Ca²⁺] (0.1 nM to 100 μM) and fitted with a non-linear regression formula (Hill sigmoid relationship) that defines the minimum [Ca²⁺] needed to activate RyR1, the half-maximal [Ca²⁺] needed to activate RyR1 maximally, and the [Ca²⁺] necessary to saturate RyR1 activity. This assay is repeated in the presence of 10 mM caffeine or 1 mM 4-cmc, or 100 μM halothane, or 10 μg/ml Buthotus hottentota venom, and other RyR1 agonists that provoke an exaggerated response in MH-susceptible RyR1 channels. The difference in the response of MH-susceptible samples is greater than 1 standard deviation above or below a mean result from a group of normal samples.

2. Direct determination of RyR1 activity

Whole homogenates or SR-enriched microsomes are reconstituted from solubilized iPSC-SkM in Muller-Rudin planar lipid bilayers to determine RyR1 activity directly. Protein is added to the cis-solution composed of 1 ml of 0.25 M cesium methanesulfonate, 10 μM CaCl₂ and 10 mM HEPES pH 7.2. The volume and composition of the trans-solution is the same. Cesium blocks K⁺ channels that may be present and that may add noise to the recordings of RyR1, whereas methanesulfonate blocks C1⁻ channels that are abundant in SR microsomes. The cis-solution is connected via an agar-KC bridge to the head stage input of a current amplifier and the trans-solution is held at virtual ground. Under these conditions, the cis and the trans sides correspond to the cytosolic and the luminal side of the channel, respectively, and direct additions of drugs or ligands of RyR1 are made. Recordings are filtered, sampled and analyzed with standard hardware (Molecular Devices, Inc) and software (pClamp program). Titration curves (Pₒ-[Ca²⁺], or Pₒ-[caffeine], or Pₒ-[halothane]) are made and the ED₅₀ for the ligand of interest is determined by fitting data points as described above.

3. [Ca²⁺]ᵢ Transients in iPSC-SkMs

Cultures of iPSC-SkMs are loaded with a fluorescent Ca²⁺ indicator (Fluo-3) and the cells are settled on the stage of a laser-scanning confocal microscope to measure electrically- or chemically-evoked [Ca²⁺]ᵢ transients. Cells are bathed in a normal Tyroid solution containing typical external ion concentrations. The amplitude and time constant (τ) of decay of the [Ca²⁺]ᵢ transient directly reflects the volume of Ca²⁺ release from RyR1 and the rate of Ca²⁺ uptake by the SR Ca²⁺ pump, respectively. Specific agonists of RyR1, increase the amplitude of the [Ca²⁺]ᵢ transient without causing major effects on τ. Both parameters of the [Ca²⁺]ᵢ transient will be measured in multiple iPSC-SkMs exposed to chemical depolarization (incremental concentrations of [K⁺]ₒ), or MH triggers (volatile anesthetics, succinylcholine), or agonists of RyR1 (caffeine, 4-chorochresol, scorpion venom) and the results plotted as described above.

4. Viability of iPSC-SkMs in the Presence of RyR1 Agonists

Cultures of iPSC-SkMs are incubated with increasing concentration of agonists of RyR1 (i.e., ryanodine, or caffeine, or B. hottentota venom) under standard incubation medium. The agonists cause persistent Ca²⁺ release, leading to Ca²⁺ toxicity and death. The magnitude and duration of Ca²⁺ release is proportional to the activity of the RyR1 channel, and RyR1 hyperactivity is a characteristic of MH-susceptible individuals. The viability of iPSC-SkMs from normal and MH-susceptible individuals is determined with Trypan blue, which is excluded from live but not dead cells. Thus, an indirect but precise determination of RyR1 activity in situ, and its response to MH triggers is obtained in multiple cells to derive a non-weighted average. Results are plotted as described above.

### 5. SR Ca²⁺ Leak-Load Relationship

“Leaky” RyR1 channels, a characteristic effect of MH mutations, refers to increased RyR1 activity at resting [Ca²⁺]. Increased SR Ca²⁺ leak is one of the parameters to ascertain the severity of the MH phenotype. Only a moderate number of cells are needed. Two methods are used in dissociated and Fluo-3 loaded iPSC-SkMs from normal and MH-susceptible individuals: first, measurements of Ca²⁺ spark rate with matched SR Ca²⁺ load indicate whether MH-susceptible cells exhibit increased Ca²⁺ release at rest, which is the function indirectly reflected in the Ca²⁺ spark rate. Normalization of spark rate with the peak of caffeine-induced Ca²⁺ release is prevents variable SR load, a critical determinant of Ca²⁺ release, from influencing results. The method of Shannon is also implemented, which estimates all RyR1-mediated Ca²⁺ leak by blocking NCX activity and measuring the resting Ca²⁺ signal before and after perfusion with tetracaine, a specific blocker of RyR1 Ca²⁺ leak. Thus, two independent and complementary methods to determine excessive Ca²⁺ leak in iPSC-SkMs from MH-susceptible individuals are provided.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in cell biology or related fields are intended to be within the scope of the following claims.

We claim:

1. A method for identifying susceptibility to malignant hyperthermia in a subject, comprising:
a) providing a sample from a subject containing one or more cells;
b) producing one or more induced pluripotent stem cells from said one or more cells;
c) generating one or more skeletal muscle myocytes from said one or more stem cells;
d) testing said one or more skeletal muscle myocytes for susceptibility to malignant hyperthermia; and
e) identifying said subject as susceptible or not susceptible to malignant hyperthermia based on the results of said testing of said one or more myocytes.

2. The method of claim 1, wherein said subject is a mammal.

3. The method of claim 2, wherein said mammal is a human.

4. The method of claim 1, wherein said sample is skin sample, a cheek swab sample, a mouthwash sample, a muscle sample, a blood sample, or a cord blood sample.

5. The method of claim 1, wherein said one or more cells is a fibroblast, a keratinocyte, an adipose cell, a nucleated blood cell, a muscle cell, or a mucosal cell.

6. The method of claim 1, wherein said producing comprises gene transfer.

7. The method of claim 1, wherein said producing comprises administration of one or more growth factors, epigenomic agonists, epigenomic antagonists, or small molecules.

8. The method of claim 1 wherein said testing comprises imaging said one or more skeletal muscle myoctes.

9. The method of claim 1, wherein said testing comprises cell electrophysiology.

10. The method of claim 9, wherein said cell electrophysiology comprises measuring one or more responses to electrical stimulation, chemical stimulation, fractional shortening or twitch tension.

11. The method of claim 1, wherein said testing comprises measuring [³H]ryanodine binding to solubilized induced pluripotent stem cell skeletal muscle myocyte homogenates.

12. The method of claim 1, wherein said testing comprises determination of RyR1 activity.

13. The method of claim 1, wherein said testing comprises measuring [Ca^{2+]}ᵢ transients in induced pluripotent stem cell skeletal muscle myocytes.

14. The method of claim 1, wherein said testing comprises measuring the viability of induced pluripotent stem cell skeletal muscle myocytes in the presence of RyR1 agonists and antagonists.

15. The method of claim 1, wherein said testing comprises measuring a sarcoplasmic Ca²⁺ leak-load relationship.

16. The method of claim 1, wherein said testing comprises the extracellular measurement of an intracellular compound comprising protons, lacatate, creatine kinase, myoglobin, troponin, or a metabolite.

17. The method of claim 1, wherein said testing comprises the intracellular measurement of an intracellular compound comprising calcium, magnesium, ATP, lactate, fatty acids and fatty acid esters.

18. The method of claim 1, wherein said testing comprises microscopy.

19. The method of claim 1, wherein said testing comprises testing in the presence and the absence of a drug.

20. The method of claim 1, further comprising testing said skeletal muscle myocytes for genetic polymorphism, copy number variation and epigenomic variation.
